(19) United States
(12) Patent Application Publication
(10) Pub. No.: US 2022/0387679 A1
(43) Pub. Date: Dec. 8, 2022

(54) ARTIFICIAL KIDNEY SYSTEM AND DEVICE

(71) Applicant: KIFFIK INC., Châteauguay (CA)

(72) Inventors: Abdeltif ESSALIK, St-Romuald (CA); Samia BENSALEM, Solothum (CH)

(21) Appl. No.: 17/770,752

(22) PCT Filed: Sep. 29, 2020
(86) PCT No.: PCT/CA2020/051298

§ 371 Date: Apr. 21, 2022

Related U.S. Application Data
(60) Provisional application No. 62/925,334, filed on Oct. 24, 2019.

(51) Int. Cl. A61M 1/14; A61B 5/145; A61M 37/00

(52) U.S. Cl. CPC A61M 1/14; A61B 5/14514; A61M 37/0015; A61B 5/150022

(57) ABSTRACT

It is provided a method and devices for detecting kidney failure and dialysis of a subject wherein the epidermis is irreversibly electroporated without generating any heat and a negative pressure is applied forming micro-conduits in the epidermis from which the interstitial fluid can be extracted, analysed and filtered to produce for example a dialysate and returned to the subject.

### TECHNICAL FIELD

It is provided a method and devices for the dialysis of the interstitial fluid by extraction and filtering the interstitial fluid of a subject.

### BACKGROUND

Dialysis is a technique which essentially allows purifying solutions. In medicine, dialysis is a method of purifying blood through a membrane.

The principle of dialysis consists in separating two solutions by a membrane to alter the chemical composition of the two solutions. Different types of membranes, semipermeable membranes (which allow only the solvent to pass through) and dialysis (pores with nanometer (nm) diameter, identical and known) are distinguished which allow the solvent and solutes to pass below a certain size. By diffusion effect (due to molecular gradient and osmosis) the small molecules will pass through the membrane, while the large molecules (often macromolecules) will be retained on one side. The main application of dialysis in the medical field concerns people whose kidneys have stopped working, temporarily (acute renal failure) or definitively (chronic end-stage renal failure). One of the products of a dialysis which refers to fluids and solutes which have crossed a membrane is called a dialysate.

In case of emergencies (e.g. in absence of functional fistula), dialysis can be initiated by a femoral, jugular or sub-keyboard venous catheter. In the event of a hemodialysis decision, an arteriovenous fistula is surgically placed by the abutment of an artery in a vein. The progressive dilatation of the fistula thus allows a much easier access to a high-flow blood channel. The onset of dialysis must wait several weeks for the fistula to develop sufficiently. Depending on the choice of the patient, more or less autonomy is possible for the preparation of the machine and the realization of the dialysis session which can be done using a machine located in the patient's home.

Peritoneal dialysis uses the peritoneum. Peritoneal dialysis is indicated mainly in the treatment of chronic end-stage renal disease. It may also be indicated in the treatment of heart failure and hypertension refractory to drug therapy. In the case of a patient with chronic renal insufficiency, the three major functions of the kidney (maintenance of the hydroelectrolyte balance influencing the regulation of blood pressure; elimination of wastes from body metabolism; and function or role as a endocrine gland) are no longer adequately assured. The peritoneal dialysis process, like the hemodialysis procedure, makes it possible to compensate for the malfunction of the first two functions.

Unlike the hemodialysis procedure which uses extracorporeal circulation equipment, peritoneal dialysis blood purification is carried out inside the body within the peritoneal cavity.

Hemodialysis is a method of purifying blood by creating an extracorporeal circulation circuit and passing it through a dialyzer. When there is severe renal insufficiency, the body is gradually charged with substances that should be eliminated by dialysis.

Continuous hemofiltration is a 24-hour dialysis technique that last several days in a row, if necessary, in the intensive care unit for patients requiring renal replacement and whose blood pressure is fragile. It cleans the blood of deleterious substances and removes excess water from the cells. It is often used in cases of septic shock, acute lung edema (water overload), anuria in cases of hemodynamic instability.

Dialysis involves bringing the blood into contact with a sterile liquid (the dialysate) which is a buffered solution through a membrane that serves as a filter. In hemodialysis this process happens outside the body and the membrane is artificial. Conversely, in peritoneal dialysis, the exchanges occur in the abdomen and the membrane is the peritoneum. In both methods, the physical phenomena involved are the same.

Hemofiltration is, like hemodialysis, a method of extra-renal purification. Its physical principle differs from that of hemodialysis: it is a convective transport. The ultra-filtered blood is collected and the collected product (the ultra-filtrate) is disposed with the waste it contains. A re-injection liquid compensates for the part of the plasma removed.

Hemofiltration allows plasma purification. Its main indication is renal supplementation but other indications are in the process of emergence (severe sepsis, SIRS, rhabdomyolysis, etc.). High-clearance (or sometimes high volume) hemofiltration is referred to when the treated volumes are greater than those required to control renal failure.

Hemodiafiltration is a derived technique that consists of both hemodialysis and hemofiltration by combining diffuse and convective transport. It is principally used in situations where the blood flow required to carry out hemofiltration is insufficient (particularly in a child).

Hepatic dialysis is a method of dialysis treatment of subjects with hepatic insufficiency and aimed at ridding the blood of toxins that accumulate in the blood due to the malfunction of the liver. This procedure has shown promise for patients with hepato-renal syndrome. It is similar to the hemodialysis used to treat renal insufficiency which mostly eliminates water-soluble toxins but does not eliminate the toxins bound to albumin that accumulate in liver failure. With extra-corporeal bio-artificial liver, it is one of two forms of artificial support of the liver by extracorporeal circulation.

Hepatic dialysis is currently considered only as a transient treatment method until transplantation or hepatic regeneration (in the case of acute liver failure) and, unlike renal dialysis, not relieve a patient over a long period of time (several months or years).

The exchange between plasma and dialysate through a membrane is dependent on two different physical phenomena which are osmosis and reverse osmosis.

Osmosis regulates the exchange of molecules in solution through a semi-permeable membrane according to the concentration gradient. That is, a given substance will be able to go from the most concentrated compartment to the less concentrated compartment, to tend towards the balance of concentrations on either side of the membrane. The speed of the exchanges will depend on the difference in concentration but also on other factors such as the pore size of the membrane, the electrical charge of the substance and that of the membrane, and the allosteric size of the substance to be exchanged. There are different dialysis membranes, each with different characteristics.

During reverse osmosis, water is displaced by the transmembrane pressure gradient, from the side where the pressure is greatest towards the side where the pressure is the lowest. The factors influencing water exchange are thus the pressure and the hydraulic coefficient of the membrane (in some way its permeability). Thanks to the combination of these two mechanisms, during a dialysis session one can simultaneously remove from the blood some substances (e.g. potassium which is provided by the diet, especially vegetables, fruit or urea which is produced by protein metabolism); add substances to the plasma that are missing (e.g. calcium which is often insufficient or bicarbonate which compensates for the acidity of the blood); and remove water that has accumulated in the body (especially if the subject is anuric, i.e. if the kidneys do not produce urine at all).

In the process of reverse osmosis, thin film composite membranes (TFC or TFM) are used. These are semipermeable membranes manufactured principally for use in water purification or desalination systems. They also have use in chemical applications such as batteries and fuel cells. In essence, a TFC material is a molecular sieve constructed in the form of a film from two or more layered materials. Membranes used in reverse osmosis are, in general, made out of polyamide, chosen primarily for its permeability to water and relative impermeability to various dissolved impurities including salt ions and other small molecules that cannot be filtered. Another example of a semipermeable membrane is dialysis tubing.

Other types of semipermeable membranes are also known and correspond for example to a cation exchange membrane (CEM), charge mosaic membrane (CMM), bipolar membrane (BPM), anion exchange membrane (AEM) alkali anion exchange membrane (AAEM) and proton exchange membrane (PEM).

The MARS system, “Molecular Adsorbents Recirculation System”, developed by Teraklin AG in Germany, is one of the most common hepatic extracorporeal dialysis systems. It consists of two separate circuits. The first circuit, composed of human serum albumin, is in contact with the patient's blood through a special semipermeable membrane (MARS®-FLUX) and has two special filters to recover the albumin after it has absorbed of the patient's blood toxins. The second circuit consists of a hemodialysis apparatus used to clean the albumin of the first circuit before it is recirculated into the first circuit. The MARS system can remove a number of toxins from the blood, such as ammonia, bile acids, bilirubin, copper, iron and phenols. Treatment is usually done on three to five 8-hour dialysis sessions.

The SPAD system (“Single Pass Albumin Dialysis”) is a simple method using albumin in renal dialysis machines without an additional perfusion system; the patient's blood circulates in a circuit with a filter identical to that used in the MARS system. The other side of the membrane is traversed by a solution of albumin circulating in countercurrent which is discarded after being passed through the filter. Hemodialysis may be performed in the first circuit by the same hollow fiber system.

The Promethée system (Fresenius Medical Care, Bad Homburg, Germany) is a new device based on the combination of albumin adsorption and high flow hemodialysis after selective filtration of albumin on a specific polysulfone filter (AlbuFlow). It was studied on a group of eleven patients with hepato-renal syndrome (acute or chronic with renal insufficiency). Treatment for two consecutive days for more than four hours significantly improved serum concentrations of conjugated bilirubin, bile acids, ammonia, cholinesterase, creatinine, urea and blood pH.

In hemodialysis, the membrane is an artificial, synthetic membrane. It is in the form of capillary fibers, the blood circulating inside the fiber, the dialysate on the outside (there is no direct contact between blood and dialysate). The capillary fibers are combined in a device called a dialyser which therefore has an inlet and an outlet for the blood and an inlet and an outlet for the dialysate. Within the dialyzer, the circulation of blood and dialysate is done in opposite directions, which optimizes the exchanges. The dialyser is continuously supplied with clean dialysate (usually dialysate feed rate of about 500 ml per minute) and blood from the patient (with a flow rate of between 150 and 500 ml per minute). The volume of blood contained in the dialyser, which varies according to the model, is approximately 100+/−20 ml. The total surface area of the dialyzer depends on the model used but ranges from 1 m² to 2.5 m².

At present, the dialysate is manufactured extemporaneously from liquid concentrate and ultrapure and sterile water. The composition of the dialysate concentrate may vary in particular at the level of the potassium concentration which may range from 1 to 3 mmol per liter and the calcium concentration which may vary from 1 to 1.75 mmol. The concentrated dialysate is diluted by the dialysis generator and mixed with a bicarbonate solution also obtained extemporaneously from a concentrated liquid powder or bicarbonate. It is therefore possible to adjust the bicarbonate content of the dialysate and the sodium content. During a routine hemodialysis session (duration of four hours) 120 liters of dialysate is produced.

Dialysis systems presently used and known are expensive and necessitate repeated sterilizations of components or the use of several syringes that have been pre-prepared with respective quantities of saline solution that must be independently introduced into the patient. This potentially allows the possible introduction of airborne contaminants into the patient or into blood from the patient, thereby contaminating the area.

There is thus still a need to be provided with new means for conducting a dialysis in a patient.

### SUMMARY

One aim of the present disclosure is to provide a method for dialysis of a subject by extracting the interstitial fluid comprising the steps of irreversibly electroporating a region of the stratum corneum of the subject without generating any heat; applying a negative pressure at or near the region of the stratum corneum of the subject being irreversibly electroporated, the combination of irreversibly electroporating the skin and applying the negative pressure forming micro-conduits in the stratum corneum; extracting the interstitial fluid of the subject; and filtering the extracted interstitial fluid of the subject forming a dialysate and a clean interstitial fluid, wherein the clean interstitial fluid is returned to the subject.

It is further provided a method of detecting kidney damage in a subject comprising irreversibly electroporating a region of the stratum corneum of the subject without generating any heat, applying a negative pressure at or near the region of the stratum corneum of the subject being irreversibly electroporated, the combination of irreversibly electroporating the skin and applying the negative pressure forming micro-conduits in the stratum corneum, extracting the interstitial fluid of the subject; and measuring the amount of a biomarker, wherein the presence of said biomarker in the interstitial fluid of said subject is indicative of kidney damage.

In an embodiment, the biomarker is at least one of creatinine and urea.

It is also provided dialysis system for non-invasively extracting interstitial fluid from a subject comprising means for irreversibly electroporating the stratum corneum by generating a non-pulsed voltage and a pulsed voltage between at least one first moving or stationary electrode and a second stationary electrode forming micro-conduits in the stratum corneum; a vacuum pump for providing a negative pressure above the stratum corneum and allowing extraction of the interstitial fluid, and at least one semipermeable membrane for filtering the extracted interstitial fluid.

In an embodiment, the extracted interstitial fluid is filtered through a semipermeable membrane.

In another embodiment, the semipermeable membrane is a cation exchange membrane (CEM), a charge mosaic membrane (CMM), a bipolar membrane (BPM), an anion exchange membrane (AEM), an alkali anion exchange membrane (AAEM) or a proton exchange membrane (PEM).

In a further embodiment, molecules are removed from the extracted interstitial fluid and passing to the dialysate.

In a further embodiment, the molecules are potassium or water or urea.

In another embodiment, substances are further supplemented to the clean interstitial fluid before being returned to the subject.

In an additional embodiment, a non-pulsed voltage and a pulsed voltage are generated between at least a first moving or stationary electrode and a second stationary electrode for electroporating the stratum corneum.

In an embodiment, the negative pressure is pulsed or continuous.

In a further embodiment, the negative pressure applied at or near the region of the stratum corneum being electroporated is monitored by a pressure sensor.

In another embodiment, the subject is an animal or a human.

In an embodiment, at least one first and second electrodes are contained in a chamber.

In another embodiment, the chamber is a separate part from the first and second electrodes used for the electroporation.

In an embodiment, the system extracts the interstitial fluid at a rate between 10 microliter/second to 200 microliter/second.

In an embodiment, the system further comprises means for returning the filtered extracted interstitial fluid to the subject.

In another embodiment, the system comprises a catheter or a network of microneedles.

In a further embodiment, the system further comprises a sensor or a plurality of sensors for analyzing a biomarker or the filtered interstitial fluid and the delivery rate of the filtered interstitial fluid of the subject.

In another embodiment, the sensor or a plurality of sensors further comprise a wired or wireless communication device.

In an embodiment, the vacuum pump is a peristaltic pump, a diaphragm pump, or a piston pump.

In another embodiment, the negative pressure generated by the vacuum pump is from about 2 kPa to about 98 kPa.

In an additional embodiment, the vacuum generated by the pump is pulsed or continuous.

### BRIEF DESCRIPTION OF THE DRAWINGS

Reference will now be made to the accompanying drawings, showing by way of illustration, a preferred embodiment thereof.

FIG. 1 illustrates an illustration of impedimetric sensor before and after the deposition of the sensing layer on the sensing electrode in accordance to an embodiment.

FIG. 2A illustrates a schematic representation of an electrical equivalent circuit between the sensing electrode and the reference electrode in accordance to an embodiment.

FIG. 2B illustrates a Nyquist plot obtained when an alternative voltage with a sinusoidal amplitude comprised between 2 to 60 mV is superimposed to the sensing electrode is maintained at a DC voltage corresponding to the rest voltage or the open circuit voltage of the electrochemical cell (impedimetric sensor) in agreement with the electrical equivalent circuit shown in FIG. 2A.

FIG. 3 illustrates a top view of the bottom of an extraction chamber as described herein in accordance to an embodiment.

FIG. 4 illustrates a planar top view of the bottom of the extraction chamber in accordance to an embodiment.

FIG. 5 illustrates a top view of the upper part of the extraction chamber in accordance to an embodiment.

FIG. 6 illustrates a top view of the upper part of the extraction chamber in accordance to an embodiment.

FIG. 7 illustrates an assembled view of one extraction chamber in conjunction with an electronic controller box as encompassed herein in accordance to an embodiment.

FIG. 8 illustrates an assembled view of one extraction chamber in conjunction with the electronic controller box in accordance to an embodiment.

FIG. 9 illustrates a top view of three extraction chambers connected in series to the same pump inside the electronic controller box in accordance to an embodiment.

It will be noted that throughout the appended drawings, like features are identified by like reference numerals.

### DETAILED DESCRIPTION

In accordance with the present description, there is provided a method for dialysis of a subject by extracting the interstitial fluid comprising the steps of irreversibly electroporating a region of the stratum corneum of the subject without generating any heat; applying a negative pressure at or near the region of the stratum corneum of the subject being irreversibly electroporated, the combination of irreversibly electroporating the skin and applying the negative pressure forming micro-conduits in the stratum corneum; extracting the interstitial fluid of the subject.

The extracted interstitial fluid of said subject can be filtered forming a dialysate and a clean interstitial fluid, wherein the clean interstitial fluid is returned to the subject.

It is also provided a method of detecting kidney damage in a subject comprising irreversibly electroporating a region of the stratum corneum of the subject without generating any heat, applying a negative pressure at or near the region of the stratum corneum of the subject being irreversibly electroporated, the combination of irreversibly electroporating the skin and applying the negative pressure forming micro-conduits in the stratum corneum; extracting the interstitial fluid of the subject; and measuring the amount of a biomarker, wherein the presence of said biomarker in the interstitial fluid of said subject is indicative of kidney damage.

In an embodiment, the biomarker is at least one of creatinine and urea.

It is described an artificial kidney system and devices. The artificial kidney system and devices encompassed herein are portable and use the interstitial fluid instead of blood to retrieve different molecules, ions, etc. or to add different molecules, ions, etc. The artificial kidney system and devices described herein use the dialysis of the interstitial fluid in a continuous fashion instead of the periodic blood dialysis.

Disclosed herein is a method which creates irreversibly hundreds of electrophilic conduits through the stratum corneum. These openings are permanent, irreversible and have a duration limited only by the duration of the extraction of the interstitial fluid. Otherwise, these openings or conduits through the stratum corneum are the subject of a controlled vacuum.

Disclosed herein is a method which creates one or more electrophilic conduit through the stratum corneum. These openings are permanent and have a duration limited only by the duration of the treatment.

The device and a method as described in the Canadian patent No. 2655017, incorporated by reference herewith in its entirety, can be used to irreversibly form one or a plurality of openings or conduits solely through the epidermis. The conduit(s) hence formed will form the means by where the interstitial fluid is extracted for further processing. No heat is generated during the process.

As encompassed herein, the term “irreversibly” is intended to describe real conduits formed through the epidermis. These conduits last if a vacuum is maintained above the part of the skin where the openings are located. These conduits have real physical dimensions such as a diameter and a depth. They are irreversible.

The openings or conduits formed when the method described herein is applied is intended to mean micro-holes through the epidermis formed following a short and controlled irreversible-electroporation as described herein. Irreversible-electroporation is the process by which and when applied on the skin of a living subject lead to the irreversible formation of micro-holes through the stratum corneum. The formed micro-holes last as long as they are needed.

More particularly, the device encompassed herein comprises means for electroporating the skin by generating a non-pulsed voltage and a pulsed voltage, and a sensor which are controlled electronically. The analysis of the signal obtained from the sensor in order to transform the information and to send the information to an alarm or a cell phone, for example, for further diffusion of the obtained information, is also controlled electronically. The irreversibly electroporation described herein of a region of the skin is done without generating any heat to preserve the connective tissue, in order to not denature molecules and collagen, eliminating any injuries to the cell scaffold and does not compromise the blood vessel matrix, which results in a clear margination of treated and non-treated areas.

As described in Canadian patent No. 2655017 the chamber for example used during the electroporation and sensing steps comprises electrodes, and more specifically, a reference electrode, a sensing electrode, and a counter electrode. Connection pads can be used to connect the electrodes to the electronic part of the device.

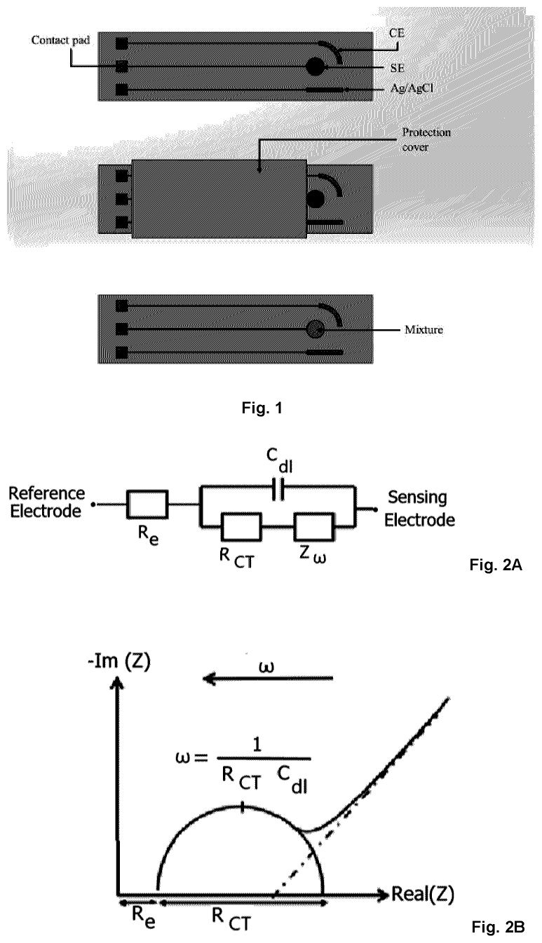

In an embodiment, the device used herein can comprise a sensing electrode as illustrated in FIG. 1. The electrode can comprise a stabilizer and a blocker of undesirable biomaterials, a promotor of adhesion such as aminosilane and nano-particles of an electronic conductor such as XC-72R on the surface of a carbon electrode (sensing electrode). The sensor electrode could be used with or without a counter-electrode.

It is described herein, measurement of an electrical parameter generated when a single frequency of an alternative voltage with a sinusoidal amplitude comprised between 2 to 60 mV is superimposed to the sensing electrode maintained at a DC voltage which corresponds to the rest voltage or the open circuit voltage of the electrochemical cell (impedimetric sensor). Such electrical parameters might include the variation of the values of the impedance (from Nyquist plot: FIGS. 2a and 2b) or the phase angle/phase shift (from Bode plot) at the interface sensing electrode/interstitial fluid.

The electrical elements as described herein (see FIG. 2A) comprise resistance of the electrolyte between the reference electrode and the sensing electrode (Rₑ) which is a pure ohmic resistance, a double layer capacitance (C_{dl}), a charge transfer resistance (R_{ct}) which is not an ohmic resistance and a Warburg diffusion element (Z_{ω}). Z_{ω} is a constant phase element (CPE), with a constant phase of 45° (phase independent of frequency) and with a magnitude inversely proportional to the square root of the frequency.

The Nyquist plot obtained when an alternative voltage with a sinusoidal amplitude comprised between 2 to 60 mV is superimposed to the sensing electrode is maintained at a DC voltage corresponding to the rest voltage or the open circuit voltage of the electrochemical cell (impedimetric sensor) in agreement with the electrical equivalent circuit shown in FIG. 2A is seen in FIG. 2B. This plot is obtained when the frequency, w, of the sinusoidal voltage has varied between 1 mHz to 100 KHz. The X axis is represented by the real values of the impedance (Real (Z)) and the Y axis is represented by the negative module of the imaginary values of the impedance (−Im (Z)) where Z is the impedance. In the plot in FIG. 2B, there is three distinct domains represented by an Ohmic resistance, Rₑ, a domain where the system is controlled by the charge transfer resistance, R_{ct}, at the interface extracted interstitial fluid and the sensing electrode with at least one time constant expressed by C_{dl}. In this disclosure, the value of the R_{ct} will increase with the increases, for example, of the concentration of the Covid-19 or its presence.

Electroporation of the skin is affected by the amplitude of the applied voltage, the shape of the applied voltage and of the electrodes, the frequency of the applied signal, the intensity of the applied current and the duration of the applied signal. The amplitude of the applied voltage is expressed in volts and can vary from 5 V to 500 V depending on the above variables and it is now not independent on the distance between the positive and the negative electrodes if this distance is comprised between 1 to 10 cm. The frequency of the applied voltage may vary from 50 μS to 7000 μS. The shape of the signal is preferred to be square but other types of signal can be used. The preferred duration is less than three minutes depending on the above cited variables, but duration above the preferred value or under the preferred value is not excluded.

Herein, irreversible electroporation is an event in which microsecond electrical pulses are applied on a living or non-living tissue, wherein the tissue is illustrated by the epidemies of a living subject, destabilizing the electrical potential across the cell membrane and resulting in irreversible nanoscale/microscale pores. Accordingly, to avoid the non-desirable Joule's effect or the generation of heat, the electrical parameters are chosen such that the cell membrane is selectively targeted without inducing thermal damage to the rest of the tissue. These parameters are illustrated hereafter in Table 1.

TABLE 1
Example of Electrical Parameters for two electrodes

| Parameters | Unit values | |
|---|---|---|
| Applied pulsed voltage | 50 to 200 | V |
| Applied DC voltage | 0.5 to 20 | V |
| Distance between two | 1 to 100 | mm |
| negative electrodes | | |
| Frequency | 50 to 7000 | μS |
| Current | 5 to 70000 | μA |
| Duty cycle/pulse | 0.05 to 27% | |
| Duration | 60 to 1700 | Seconds |
| Time between each 60 s | 15 to 20 | s |
| of electroporation | | |

| | |
|---|---|
| Measurement of the resistance | after each 60 Sec. between |
| | negative electrodes |

Due to its non-heating nature, the irreversible electroporation described herein does not affect the connective tissue nor does it denature molecules and collagen, eliminating any injuries to the cell scaffold and does not compromise the blood vessel matrix, and that the irreversible electroporation results in a clear margination of treated and non-treated areas. The generation of heat during the electroporation process has many adverse effects such as sensation of pain, destruction of the tissue inside the electroporated surface area and around this surface area.

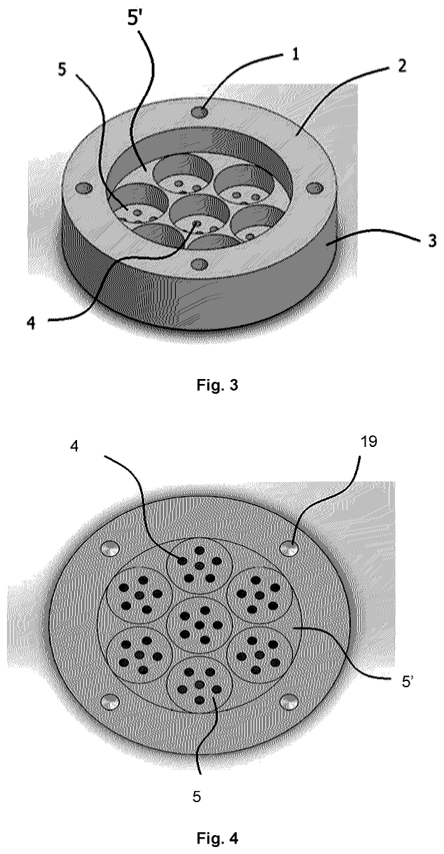

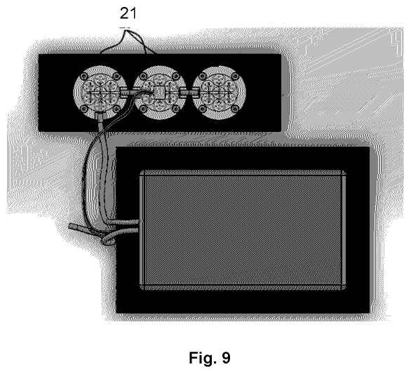

An embodiment of the extraction chamber of the device encompassed herein is illustrated in FIGS. 3 to 9, showing a typical application of the method and is designed to be used for occasional or continuous sampling of the extracted transdermal fluid or simply its continuous extraction as encompassed herein. The sampling method could be manual or automated. The device comprises an insulating body (see FIG. 3; 5, 5′, 2 and 3) maintained in contact with the skin through the epidermis using an adhesive or any other suitable sealing product or maintaining method 26.

The extraction chamber comprises at least one hole 1 to receive a screw 19, a body 3 of the extraction chamber is made of a polymer body 2 with holes 4, wherein negative solid electrodes passes through said holes 4 to be in contact with the skin. A thin layer membrane 5 of 50 to 200 μm is used to keep the skin in place and to avoid its suction. A further thicker membrane 5′ of 2 to 5 mm is used to separate the different extraction mini-chamber.

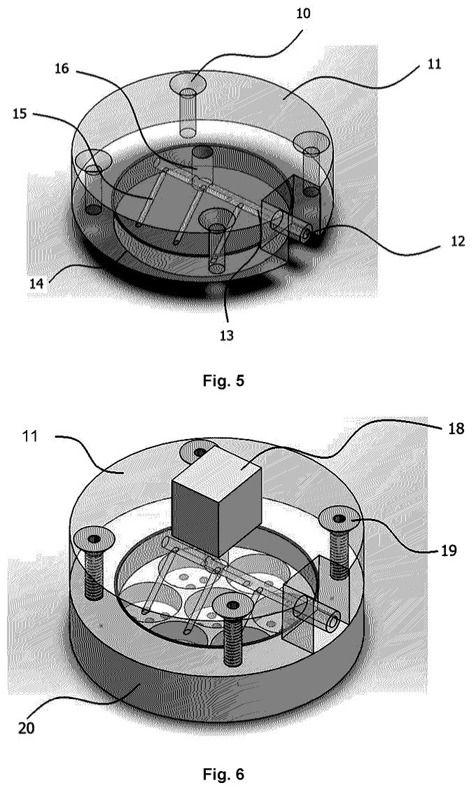

As seen in FIG. 5, in the upper part of the extraction chamber, a polymer is used to make the upper part 11 of the extraction chamber comprising the receiving ends 10 of holes that receive screws 19. An outlet of the extraction chamber 12 is connected to the inlet of the pump. A collection micro-conduit 13 serves to collect the extracted native interstitial fluid from the micro-conduits 15 and serves, also, to accommodate the pressure sensor 16. The connection between the bottom and the upper part of the extraction chamber is made by the platform 14.

The extraction chamber 21 can be made completely from a polymeric material, a combination of a polymeric material or any other material that are electrically non-conductive or insulating.

As seen in FIG. 6, a cape 18 is used to protect a pressure sensor, wherein screws 19 to fix the bottom to the upper part 20 of the extraction chamber.

The device can be covered by a material such as a capsule or an adhesive in order to facilitate its use, particularly in a situation where a liquid can surround the extraction chamber 21. The extraction chamber 21 can include a sealed hole so that the collected freshly extracted fluid can be analyzed remotely from the device. Furthermore, in order to increase the fixture of the extraction chamber 21 as described herein, a non-allergenic material such as a strap can be used to stabilize the contact between the device and the skin.

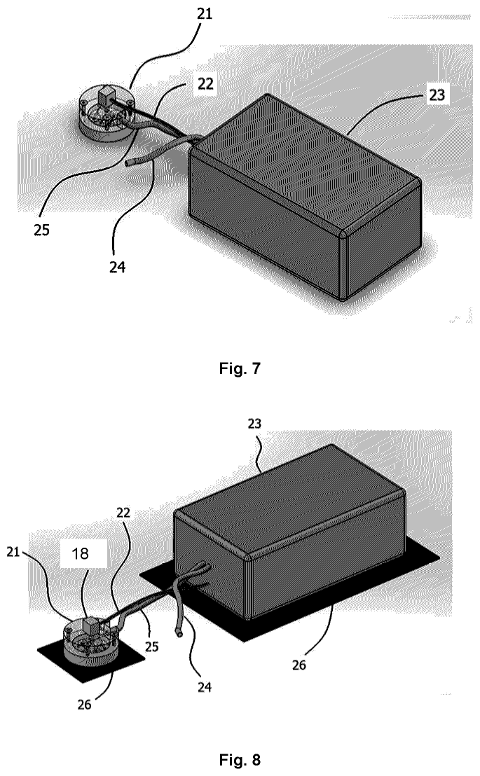

FIG. 7 illustrates in accordance to an embodiment an assembled view of one extraction chamber 21 in conjunction with an electronic controller box 23. The extraction chamber 21, is connected to the electronic controller box 23 containing the pump enclosed therein by an electrical cable 22 connecting the pressure sensor to the electronic controller box 23, wherein an outlet 24 and inlet 25 of the pump enters said electronic controller box 23.

Before the application of the extraction chamber 21 on the skin, the skin can be gently cleaned by any known chemicals used in medicine to clean such skin or simply using a soap and/or water. Preferably, any chemicals that evaporate after cleaning are used such that they do not leave any residue at the surface of the skin. Moreover, the chemicals or the soap should not induce any allergenic reaction of the skin nor modify the structure of the skin. Furthermore, in some cases the use of Povidone-iodine (PVP-I), also known as iodopovidone must be used for skin disinfection before the electroporation and the installation of the extraction chamber 21 and after the end of the extraction of the native interstitial fluid. As soon as the skin is gently cleaned or preferably disinfected, the extraction chamber 21 is attached on the top of the cleaned or disinfected skin by the help of the non-allergenic material such as the adhesive 26 or the strap or bracelet (FIG. 7). The adhesive 26 may or may not be based on an electrically conductive adhesive.

In an embodiment, pumping conduits can be in contact with a least one mini-sampling chamber and the complete extraction chamber 21, where the chosen sampling device shall be able to collect samples of the extracted transdermal fluid. The collection can be carried out, for example through a micros conduit which allows for continued extraction of the native interstitial fluid of a sealed sampling chamber 21. The sampling chambers, pumping conduits and extraction chamber 21 may or may not be isolated from a pump for example by a check valve or a one-way valve, thus creating a discontinuity between the analyzed fluid and the freshly extracted fluid which is to be analyzed. The check valve can be any means that creates or help to achieve a discontinuity between the analyzed fluid and the freshly extracted fluid such check valve could be the pump itself when it is a peristaltic pump. The extraction is continuously carried out by the application of a controlled negative pressure that is applied in the conduit system comprised namely of the extraction chamber 21, the extraction or the pumping conduit and the sampling chambers. The controlled negative pressure is maintained by the action of the pump and the level of the negative pressure is continuously monitored and controlled using a pressure sensor or pressure switch, which could be located before or after the check valve.

The negative pressure is attained by activating the pump in an on/off fashion or in a continuously modulated fashion. The actual value of the negative pressure can be controlled using the pressure sensor or a pressure switch. The pump and the pressure sensor can be under the control of a microcontroller that shall continuously or discontinuously monitor the actual pressure in the device from reading the actual status of the pressure sensor or the pressure switch and shall accordingly activate or deactivate the operation of the pump in a continuous, discontinuous or modulated fashion in order to achieve the desired negative pressure. A temperature sensor can also be incorporated in order to improve the efficiency of the device and to correct the reading of the biosensor or the plurality of biosensors if needed. Furthermore, for example, a flow sensor could be installed between the outlet of the extraction chamber 21 and the pump in order to inform the user that the process of the extraction is going well.

The vacuum is generated inside the chamber 21 between the skin and the pump in order to improve the adhesion, the continuous extraction of the transdermal fluid, the circulation of the extracted fluid and, consequently, the continuous monitoring of one analyte or a plurality of analytes in the extracted native interstitial fluid or simply the collection of the native interstitial fluid. Preferably, the vacuum pump can provide a vacuum that will provide enough suction to stretch the portion of the skin in the region from which the sample of interstitial fluid is to be extracted. As the suction provided by the vacuum pump is stretching the appropriate portion of the skin, the suction provided by the vacuum pump also causes the stretched portion to become filled with interstitial fluid. A vacuum pump that is suitable for the device defined herein can be a peristaltic pump, a diaphragm pump, a piston pump, a rotary vane pump, or any other pump that will perform the required functions set forth previously. Typically, the vacuum pump preferably employs a self-contained permanent magnet DC motor. Vacuum pumps that are suitable for this invention are well-known to those of ordinary skill in the art and are commercially available. The vacuum pump is preferably capable of providing a differential pressure down to about 0.90 atm, and is more preferably operated at from about 0.99 atm and 0.2 atm. The vacuum provided by the vacuum pump can be continuous or pulsed. It is preferred that the applied vacuum does not cause irreversible damage to the skin. It is preferred that the applied vacuum does not produce bruises and discolorations of the skin that persist for several weeks. It is also preferred that the level of applied vacuum and the duration of application of the vacuum do not be so excessive that it causes the dermis to separate from the epidermis, which results in the formation of a blister filled with fluid.

The irreversible electroporation of the skin is carried out by the application of a pulsed voltage between two electrodes or a plurality of electrodes forming a network of electrodes. As an illustration of the art herein, the excitation part of the process is carried out using at least two electrodes placed in contact with the exposed portion of the skin which is the negative electrode and the positive electrode which is used also as an adhesive. One of the excitation electrodes (negative electrode or electroporating electrode) is maintained in contact with the exposed portion of the skin during the non-heating irreversible electroporation.

The target result of the irreversible electroporation herein is an effective and non-invasive electropermeabilization of the epidermis leading to permanent micro-opening(s) as long as the suction is applied, without any pain felling and which can permit a continuous transdermal fluid extraction for as long as needed. Herein, the transdermal fluid is the native interstitial fluid. A continuous fluid extraction is intended herein to refer to a continuous flow of the interstitial liquid through the skin, including the opening(s) in the epidermis, into the extraction chamber 21.

The system or devices described herein further comprises semipermeable membrane consisting of a thin layer of material that contains holes of various sizes, or pores. Smaller solutes and fluid pass through the membrane, but the membrane blocks the passage of larger substances (for example, large molecules such as large proteins). This replicates the filtering process that takes place in the kidneys, when the blood enters the kidneys and the larger substances are separated from the smaller ones in the glomerulus.

The exchange between the continuous extracted interstitial fluid and the dialysate through a membrane is dependent on two different physical phenomena which are osmosis and reverse osmosis.

Osmosis regulates the exchange of molecules between the continuous extracted interstitial fluid and the dialysate through a semi-permeable membrane according to the concentration gradient. Accordingly, a given substance will be able to go from the most concentrated compartment (from the continuous extracted fluid into the less concentrated compartment (dialysate and vice-versa) to tend towards the balance of concentrations on either side of the membrane. The speed of the exchanges will depend on the difference in concentration but also on other factors such as the pore size of the membrane, the electrical charge of the substance and that of the membrane, and the allosteric size of the substance to be exchanged.

Furthermore, described here is the use of reverse osmosis where water must be displaced by the transmembrane pressure gradient from the continuous extracted interstitial fluid to the dialysate.

Accordingly, as described herein, water will pass from the side where the pressure is greatest towards the side where the pressure is the lowest. The factors influencing water exchange are the pressure and the hydraulic coefficient of the membrane (in some way its permeability).

Described herein is the combination of the osmosis and the reverse osmosis, during the dialysis of the continuous extracted interstitial fluid. This combination will lead simultaneously to the removal from the continuous extracted interstitial fluid some substances which are in for example, potassium which is provided by the diet, especially vegetables, fruit or urea which is produced by protein metabolism; or add substances to the continuous extracted interstitial fluid that are missing, such as calcium which is often insufficient or bicarbonate which compensates for the acidity of the blood and the interstitial fluid. Furthermore, water can be removed that has accumulated in the body (especially if the subject is anuric).

In an embodiment, the semipermeable membrane is a cation exchange membrane (CEM), a charge mosaic membrane (CMM), a bipolar membrane (BPM), an anion exchange membrane (AEM), an alkali anion exchange membrane (AAEM) and a proton exchange membrane (PEM).

A crucial problem encountered in liver failure is the accumulation of toxins that are no longer eliminated by the failing liver. Based on this hypothesis, the removal of albumin-bound lipophilic substances such as bilirubin, bile acids, aromatic amino acid metabolites, medium chain fatty acids and cytokines should be beneficial for improvement of a patient with hepatic impairment. This has led to the production of artificial filters and adsorption devices.

Accordingly, it is provided a mean for the dialysis of continuous extracted interstitial fluid. Described herein is the dialysis of continuous extracted interstitial based on the principles of the diffusion of some molecules present in the extracted interstitial fluid and the ultrafiltration of fluid across a semi-permeable membrane. Thus, it is encompassed the diffusion of some molecules present in the extracted interstitial fluid move from an area of high concentration to an area of low concentration.

In an embodiment, the solution of low concentration constitutes the dialysate.

The system provided herein is for the continuous extraction of interstitial fluid with a rate preferably comprised between 10 microliter/second to 200 microliter/second.

In an embodiment, the system described herein is for the continuous extraction of interstitial fluid which can be placed in any part of the body if the user will remain comfortable.

The system is intended to remain comfortable and will remain in place for a period exceeding one week and can reach six months.

In a further embodiment, the product of the dialysis of the continuous extracted fluid can be returned to the body by the use for example of a catheter or a network of microneedles which could be placed anywhere in the body, preferably in peritoneal part of the body or the interstitial space.

It is also encompassed that the system described herein comprises a mean to monitor the quality of the product of the dialysis of the continuous extracted interstitial fluid before its injection in the body.

Kidney damage is marked by the rise in two important chemical substances in the blood—creatinine and urea. Generally, urea accumulation in blood serum of kidney failure patients increases due to the degradation of food and tissues such as muscle. The high level of urea in blood leads to the sickness of the body if not removed by a healthy kidney or by hemodialysis to a normal value. As seen in Example I, the value of urea before and after hemodialysis does not return to a normal value, its value remains very high and worst it is higher in the interstitial fluid before and after the hemodialysis. It is well known that the site of the production and damage of urea and creatinine is the interstitial space. Therefore, the method described herein provides a meant to perform a continuous dialysis of the interstitial fluid where the most damaging molecules due to the kidney failure are found. The continuous dialysis of the interstitial fluid disclosed herein will maintain the values of the majority of the molecules accumulated in the body due to the renal failure very close to the homeostatic values.

### Example I

### Analysis of Analytes in the Interstitial Fluid and the Blood of a Volunteer Before and After the Hemodialysis

In a volunteer, a surface area has been identified by an adhesive on both arms of the same volunteer for comparative purposes, wherein both surface areas were dimensionally equal. One of the surfaces delimited by the adhesive was electroporated irreversibly according to the method described herein.

During electroporation a micro-thermistor was inserted into the space where the electroporation took place. This space is delimited by a micro-chamber which isolates it from the ambient atmosphere. The purpose of using the micro-thermistor was to measure the temperature during the irreversible electroporation if heat was generated.

The temperature before, during and after the electroporation remained the same and did not change from its initial value which was 30° C.

Table 1 below provides measured analytes in the interstitial fluid and the blood of the volunteer before and after the hemodialysis. The volunteer has been performing dialysis of his blood for a few years. Moreover, the volunteer performs the hemodialysis every three days. The results in the Table 1 demonstrated efficient measurement of creatinine and urea by the process described herein.

TABLE 1
The values of some biomarkers present in the interstitial
fluid and the blood of a volunteer before and
after the hemodialysis of his blood.

| | Interstitial | Interstitial | Blood | Blood |
|---|---|---|---|---|
| | fluid before | fluid after | before | after |
| | the hemo- | the hemo- | the hemo- | the hemo- |
| Biomarker | dialysis | dialysis | dialysis | dialysis |
| Alkaline reserves | 19.5 | 20.2 | 22.3 | 29.4 |
| (Bicarbonate) mM/L | | | | |
| Urea mM/L | 28.8 | 20.1 | 22.1 | 14.1 |
| Creatinine mg/L | 110.11 | 64.79 | 101.82 | 54.34 |
| CPK UI/L | 144.0 | 74.0 | 123.0 | 116.0 |
| Glucose g/L | 1.28 | 1.29 | 0.93 | 1.42 |
| LDH UI/L | 243 | 243 | 198 | 179 |
| Sodium mM/L | 122.8 | 124.4 | 128.0 | 137.6 |
| Potassium mM/L | 5.90 | 5.53 | 5.48 | 4.59 |
| Chloride ion mM/L | 88.0 | 92.8 | 84.4 | 88.9 |
| Transaminases | 19.4 | 16.4 | 15.3 | 14.9 |
| (ASAT) UI/L | | | | |
| Transaminases | 6.6 | 5.1 | 7.7 | 8.2 |
| (ALAT) UI/L | | | | |
| Total Cholesterol | 0.38 | 0.21 | 1.58 | 1.4 |
| Total g/L | | | | |
| Cholesterol HDL g/L | 0.10 | 0.10 | 0.30 | 0.30 |
| Cholesterol LDL g/L | 0.21 | 0.06 | 1.02 | 0.86 |
| Triglycerides g/L | 0.33 | 0.24 | 1.30 | 1.34 |
| PSA ng/mL | 0.356 | 0.327 | 0.815 | 0.784 |
| Albumin g/L | 18.10 | 12.10 | 47.00 | 43.00 |
| Gamma-GT UI/L | 8.0 | 5.0 | 33.0 | 39.0 |
| C Reactive Protein | 0.01 | 0.01 | 0.89 | 0.95 |
| mg/L | | | | |

Table 2 shows the value of some biomarkers of a healthy person. As can be seen from the Tables 1 and 2, the present method provides an effective means to measure concentrations of key biomarkers to assess the renal failure and the effectiveness of the hemodialysis. The values shown in Table 2 were obtained from a volunteer research subject as part of a three-day study. The volunteer was under intense physical effort before the beginning of the extraction of his interstitial fluid. The blood of the volunteer was obtained three weeks after the extraction of the interstitial fluid.

TABLE 2
Value of biomarkers of an healthy person

| Biomarkers in subject's | | | | | |
|---|---|---|---|---|---|
| interstitial fluid (extracted | | | | | |
| via the method described) | | | Biomarkers in subject's blood | | |
| General Biochemistry | | | General Biochemistry | | |
| Urea | 7.5 | mM/L | Urea | 4.3 | mM/L |
| Creatinine | 73 | μM/L | Creatinine | 93 | μM/L |
| Sodium | 161 | mM/L | Sodium | 138 | mM/L |
| Potassium | 4.9 | mM/L | Potassium | 3.8 | mM/L |
| Chlorides | 126 | mM/L | Chlorides | 102 | mM/L |
| Total CO₂ | 26 | mM/L | Total CO₂ | 26 | mM/L |
| Total calcium | 1.38 | mM/L | Total calcium | 2.52 | mM/L |
| Ionized calcium | 0.67 | mM/L | Ionized calcium | 1.22 | mM/L |
| Total Bilirubin | 5 | μM/L | Total Bilirubin | 12 | μM/L |
| Total Proteins | 35 | g/L | Total Proteins | 73 | g/L |
| Albumin | 25 | g/L | Albumin | 48 | g/L |
| AST | 48 | U/L | AST | 28 | U/L |
| ALT | 9 | U/L | ALT | 19 | U/L |
| GGT | 5 | U/L | GGT | 11 | U/L |
| Alkaline | 18 | U/L | Alkaline | 37 | U/L |
| Phosphatase | | | Phosphatase | | |
| Lactic acid | 5.2 | mM/L | Lactic acid | 2.6 | mM/L |
| C-reactive protein | <0.2 | mg/L | C-reactive protein | <0.2 | mg/L |
| Erythropoiesis | | | Erythropoiesis | | |
| Vitamin B12 | 341 | pM/L | Vitamin B12 | 310 | pM/L |
| Folic acid | >45.4 | nM/L | Folic acid | 25.4 | nM/L |
| Specific proteins: | | | Specific proteins: | | |
| Immunoglobulins | | | Immunoglobulins | | |
| IgG | 3.12 | g/L | IgG | 7.45 | g/L |
| IgA | 0.40 | g/L | IgA | 1.17 | g/L |
| IgM | 0.31 | g/L | IgM | 1.17 | g/L |
| Endocrinology | | | Endocrinology | | |
| Cortisol | 115 | nM/L | Cortisol | 565 | nM/L |

While the disclosure has been described in connection with specific embodiments thereof, it will be understood that it is capable of further modifications and this application is intended to cover any variations, uses, or adaptations, including such departures from the present disclosure as come within known or customary practice within the art, and as may be applied to the essential features hereinbefore set forth, and as follows in the scope of the appended claims.

1-26. (canceled)

27. A dialysis system for non-invasively extracting interstitial fluid from a subject comprising:
an electroporator generating for irreversibly electroporating the stratum corneum by generating a non-pulsed voltage and a pulsed voltage between at least one first moving or stationary electrode and a second stationary electrode forming micro-conduits in the stratum corneum;
a vacuum pump for providing a negative pressure above the stratum corneum and allowing extraction of said interstitial fluid; and
at least one semipermeable membrane for filtering the extracted interstitial fluid.

28. The system of claim 27, wherein the semipermeable membrane is a cation exchange membrane (CEM), a charge mosaic membrane (CMM), a bipolar membrane (BPM), an anion exchange membrane (AEM), an alkali anion exchange membrane (AAEM) or a proton exchange membrane (PEM).

29. The system of claim 27, wherein said at least one first and second electrodes are contained in a chamber.

30. The system of claim 29, wherein the chamber is a separate part from the first and second electrodes used for the electroporation.

31. The system of claim 27, wherein the system extracts the interstitial fluid at a rate between 10 microliter/second to 2000 microliter/second.

32. The system of claim 27, further comprising means for returning the filtered extracted interstitial fluid to the subject.

33. The system of claim 32, wherein the system comprises a catheter or a network of microneedles.

34. The system of claim 27, further comprising a sensor or a plurality of sensors for analyzing a biomarker or a plurality of biomarkers.

35. The system of claim 34, wherein the biomarker is at least one of pH, creatinine and urea.

36. The system of claim 27, further comprising a sensor or a plurality of sensors for analyzing the filtered interstitial fluid and the delivery rate of the filtered interstitial fluid to the subject.

37. The system of claim 36, wherein said sensor or a plurality of sensors further comprise a wired or wireless communication device.

38. The system of claim 27, wherein said vacuum pump is a peristaltic pump, a diaphragm pump, or a piston pump.

39. The system of claim 27, wherein said negative pressure generated by the vacuum pump is from about 2 kPa to about 98 kPa.

40. The system of claim 27, wherein said vacuum generated by the pump is pulsed or continuous.

41. A method for dialysis of a subject by extracting the interstitial fluid comprising the steps of:
irreversibly electroporating a region of the epidermis of the subject without generating any heat using the device of claim 27;
applying a negative pressure at or near the region of the epidermis of the subject being irreversibly electroporated, the combination of irreversibly electroporating the skin and applying the negative pressure forming micro-conduits in the epidermis;
extracting the interstitial fluid of said subject; and
filtering the extracted interstitial fluid of said subject forming a dialysate and a clean interstitial fluid,
wherein the clean interstitial fluid is returned to the subject.

42. The method of claim 41, wherein the extracted interstitial fluid is filtered through a semipermeable membrane.

43. The method of claim 41, wherein molecules are removed from the extracted interstitial fluid and passing to the dialysate.

44. The method of claim 43, wherein said molecules is potassium, water or urea.

45. The method of claim 41, wherein a non-pulsed voltage and a pulsed voltage are generated between at least a first moving or stationary electrode and a second stationary electrode for electroporating the stratum corneum.

46. The method of claim 41, further comprising the step of measuring the amount of a biomarker in said interstitial fluid of said subject.
